(19) United States
(12) Patent Application Publication
(10) Pub. No.: US 2013/0190615 A1
(43) Pub. Date: Jul. 25, 2013

(54) System for 3D Visualization of Radio-Opaque Embolic Materials Using X-ray Imaging

(71) Applicants: Kevin Royalty, Fitchburg, WI (US); Frederik Bender, Erlangen (DE); Maria Lydia Sarmiento, New York, NY (US)

(72) Inventors: Kevin Royalty, Fitchburg, WI (US); Frederik Bender, Erlangen (DE); Maria Lydia Sarmiento, New York, NY (US)

(21) Appl. No.: 13/690,247

(22) Filed: Nov. 30, 2012

Related U.S. Application Data
(60) Provisional application No. 61/589,471, filed on Jan. 23, 2012.

(51) Int. Cl. A61B 6/00; A61B 6/02

(52) U.S. Cl. CPC A61B 6/481; A61B 6/02; A61B 6/5294; A61B 6/466
USPC 600/431

(57) ABSTRACT

An image data processor automatically identifies individual picture elements representing embolic material in first and second 2D X-ray images in response to a luminance intensity value of the picture elements exceeding a threshold. The image data processor also automatically identifies individual volume elements in a 3D X-ray image dataset corresponding to the identified individual picture elements by, for an individual picture element, detecting intersection of a projected line with one or more volume elements in the 3D image dataset representing vessels. The projected line substantially passes from the individual picture element to an X-ray radiation source. The display processor initiates generation of data representing a display image showing the identified individual volume elements representing embolic material, with enhanced visualization.

This is a non-provisional application of provisional application serial No. 61/589,471 filed Jan. 23, 2012, by K. Royalty et al.

### FIELD OF THE INVENTION

This invention concerns a system for three dimensional (3D) visualization of embolic material using X-ray radiation imaging by processing elements of two dimensional (2D) images and a 3D image dataset to identify individual volume elements representing embolic material.

### BACKGROUND OF THE INVENTION

Known C-arm based bi-plane X-ray imaging systems are mechanically constrained to a small range of angulations and views. For deployment of radio-opaque embolic materials and agents, it is desirable that working projections in imaging plane A and plane B provide visibility of the extent of embolic deployment. This is necessary to prevent embolization of structures downstream of the anatomical embolic vascular targets (e.g. AVMs—Arterio-Venous Malformations) or refluxing retrograde down a feeding artery. Physicians often have to move their eyes quickly back and forth from plane A to plane B to make sure the embolic material has reached targeted portions of patient anatomy. In known systems a physician relies on the 2D fluoroscopic images to visualize embolic deployment.

In known systems for visualizing liquid embolic material used to treat vascular malformations a physician is constrained to a mechanical range of an X-ray system C-arm. This limits the range of working projections that a physician can use during embolic deployment. Due to this limitation, a physician is often required to use simultaneous bi-plane fluoroscopic images to evaluate the deployment. This is a difficult task, as it requires the physician to continually switch back and forth as the liquid embolic material is deployed. During long cases, this can be tiring for a physician. Since embolizations are often performed in stages, using fluoroscopic views also becomes difficult due to the radio-opaque liquid embolic cast that remains from a previous treatment. This leads a physician to use a roadmapping feature available in most modern angiography systems.

FIG. 1 shows a roadmap comprising a subtracted Fluoroscopy image indicating AVM Embolization. Using the roadmapping feature allows a physician to subtract out the original cast, but offers poor contrast resolution and obscures the visualization of the liquid embolic material. In addition, since a roadmap mask is often reset several times through a procedure, it becomes difficult to distinguish what has been done during the current procedure, and what was embolized in the previous procedure (FIG. 1). It also provides no visual guide as to the extent of the embolized volume of the target relative to its total original volume.

Cerebral arteriovenous malformations (AVMs) and fistulas are complex vascular abnormalities that are difficult for physicians to treat and often require multiple treatment options to reach a satisfactory endpoint. Known systems provide treatment options, often used in combination including surgical removal, where the surgeon invasively excises the AVM from the brain. Another option is radiosurgery, where focused radiation targets the AVM with the aim of obliterating a tangle of vessels that make up the AVM. A further option is arterial-based embolization of the AVM. This occurs when small particles or a thick radio-opaque glue-like substance is slowly injected into the vessels that feed the AVM until flow through the AVM is sufficiently halted. In many cases, embolization is often done in stages and can be used as a pre-treatment option to increase the effectiveness of radiosurgery or reduce the bleeding complications for surgical treatment.

Endovascularly trained radiologists and surgeons typically treat patients using a highly viscous liquid embolization suspension that is radio-opaque under X-ray. Typically a bi-plane angiography system is used such that the physicians can watch the delivery of the material from several angles to better understand how the embolization material is flowing with the blood vessels. A physician guides a small catheter to the blood vessels that are directly feeding the AVM, and slowly begin injecting embolic material under X-ray. It is desirable to have a smooth slow delivery, as applying too much pressure can result in either reflux of the material retrograde to other parts of the brain, or pushing the material through the AVM and into a major cerebral vein. Either of these outcomes can result in stroke in the patient and be immediately life-threatening with little opportunity to correct the problem. Further, during a procedure, the physician is burdened by having to move his eyes back and forth between the two available X-ray projections to make sure the embolic material only flows to the desired location. A system according to invention principles addresses these deficiencies and related problems.

### SUMMARY OF THE INVENTION

A system provides real-time 3D visualization of radio-opaque embolic material using bi-plane fluoroscopic images, for example, by backprojecting high contrast pixels representing opaque embolic material to a 3D coordinate space or by using two fluoroscopic projections backprojected into 3D space, for fast, real-time 3D visualization of radio-opaque embolic material. A system for three dimensional (3D) visualization of embolic material using X-ray radiation imaging, includes a repository of information, an image data processor and a display processor. The repository of information includes a 3D image dataset representing an anatomical volume of interest including vessels for receiving X-ray absorbent embolic material and first and second 2D images acquired in different planes and showing vessels including X-ray absorbent embolic material. The image data processor automatically identifies individual picture elements in the first and second 2D images representing embolic material in response to a luminance intensity value of the picture elements exceeding a threshold. The image data processor also automatically identifies individual volume elements in the 3D image dataset corresponding to the identified individual picture elements. The display processor initiates generation of data representing a display image showing the identified individual volume elements representing embolic material, with enhanced visualization.

### BRIEF DESCRIPTION OF THE DRAWING

FIG. 1 shows a roadmap comprising a subtracted Fluoroscopy image indicating AVM Embolization.

FIG. 2 shows a system for three dimensional (3D) visualization of embolic material using X-ray radiation imaging, according to invention principles.

FIG. 3 shows a flowchart of a process for three dimensional (3D) visualization of embolic material using X-ray radiation imaging, according to invention principles.

FIG. 4 shows a flowchart of a workflow process for three dimensional (3D) visualization of embolic material using X-ray radiation imaging, according to invention principles.

FIG. 5 illustrates identifying individual volume elements in a 3D image dataset corresponding to identified individual picture elements in 2D images representing embolic material by, for an individual picture element, detecting intersection of a projected line with one or more volume elements in the 3D image dataset, according to invention principles.

FIG. 6 shows a flowchart of a process employed by a system for three dimensional (3D) visualization of embolic material using X-ray radiation imaging, according to invention principles.

### DETAILED DESCRIPTION OF THE INVENTION

A system provides real-time 3D visualization of radio-opaque embolic materials using bi-plane fluoroscopic images, for example. High-contrast radio-opaque embolic material is segmented in planar fluoroscopic or roadmap images (subtracted fluoroscopic images) by thresholding, and high contrast pixels are back-projected to a 3D coordinate space. Alternatively, two fluoroscopic projections are back-projected into 3D space, and thresholding applied. Once in the 3D coordinate space, a 3D DSA (Digital Subtraction Angiography) angiogram of the vessel anatomy is used to constrain back projected pixels to a previously rendered vessel tree, and the voxels that have an intersection from both planes are colored to show where the embolic material has been delivered. This allows for fast, real-time 3D visualization of the deployment of radio-opaque embolic material using a previously acquired 3D DSA as the basis for rendering constraints. Embolization comprises the therapeutic introduction of a substance (embolic material) into a vessel in order to occlude it.

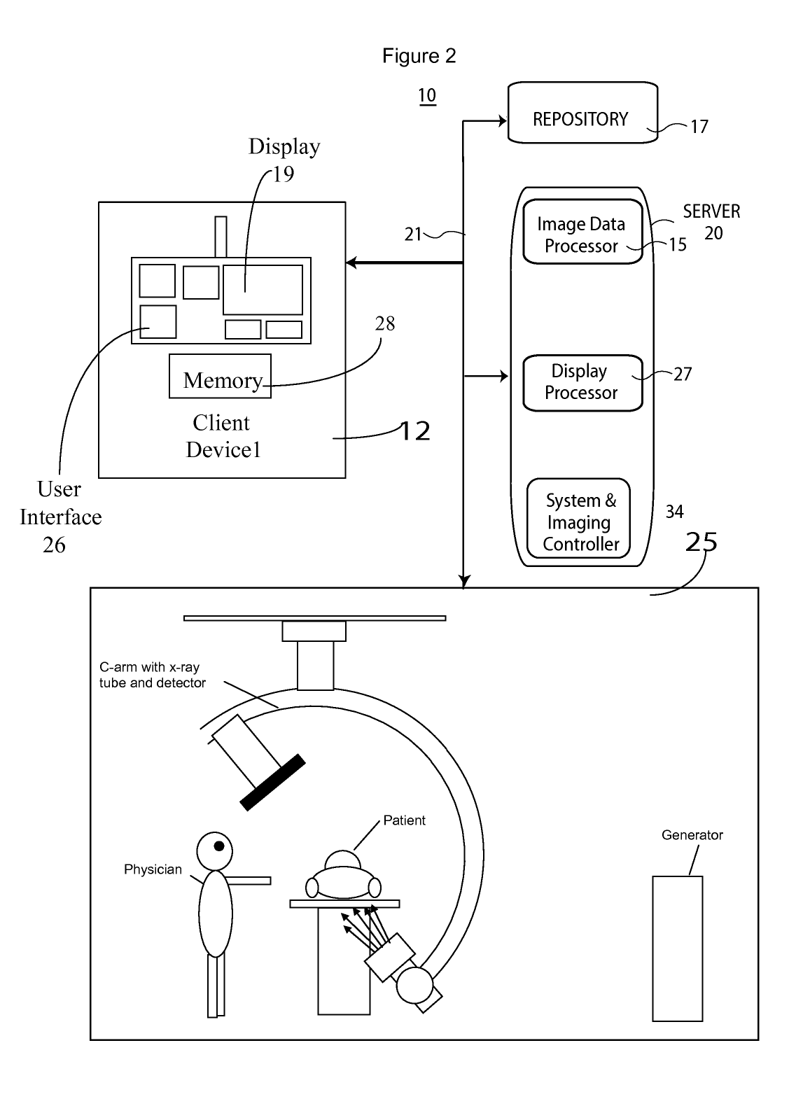

FIG. 2 shows system 10 for three dimensional (3D) visualization of embolic material using X-ray radiation imaging. The system advantageously provides real-time 3D image information that shows how embolic material is migrating through blood vessels. The blood vessel anatomy (acquired from a vascular 3D image volume dataset) and real-time measurements of X-ray attenuation from two concurrently acquired X-ray projections are advantageously geometrically calibrated to the system coordinate space.

System 10 includes one or more processing devices (e.g., workstations, computers or portable devices such as notebooks, Personal Digital Assistants, phones) 12 that individually include memory 28, a user interface 26 enabling user interaction with a Graphical User Interface (GUI) and display 19 supporting GUI and medical image presentation in response to predetermined user (e.g., physician) specific preferences. System 10 also includes at least one repository 17, server 20, and imaging device 25. Server 20 includes image data processor 15, Display processor 27 and system and imaging control unit 34. System and imaging control unit 34 controls operation of one or more imaging devices 25 for performing image acquisition of patient anatomy in response to user command Imaging devices 25 may comprise a mono-plane or biplane X-ray imaging system. The units of system 10 intercommunicate via network 21. At least one repository 17 stores X-ray medical images and studies for patients in DICOM compatible (or other) data format. A medical image study individually includes multiple image series of a patient anatomical portion which in turn individually include multiple images. Repository 17 includes a 3D image dataset representing an anatomical volume of interest including vessels for receiving X-ray absorbent embolic material and first and second 2D images acquired in different planes and showing vessels including X-ray absorbent embolic material.

Image data processor 15 automatically identifies individual picture elements in the first and second 2D images representing embolic material in response to a luminance intensity value of the picture elements exceeding a threshold. Processor 15 automatically identifies individual volume elements in the 3D image dataset corresponding to the identified individual picture elements by, for an individual picture element, detecting intersection of a projected line with one or more volume elements in the 3D image dataset representing vessels. The projected line substantially passes from the individual picture element to an X-ray radiation source. Display processor 27 initiates generation of data representing a display image showing the identified individual volume elements representing embolic material, with enhanced visualization.

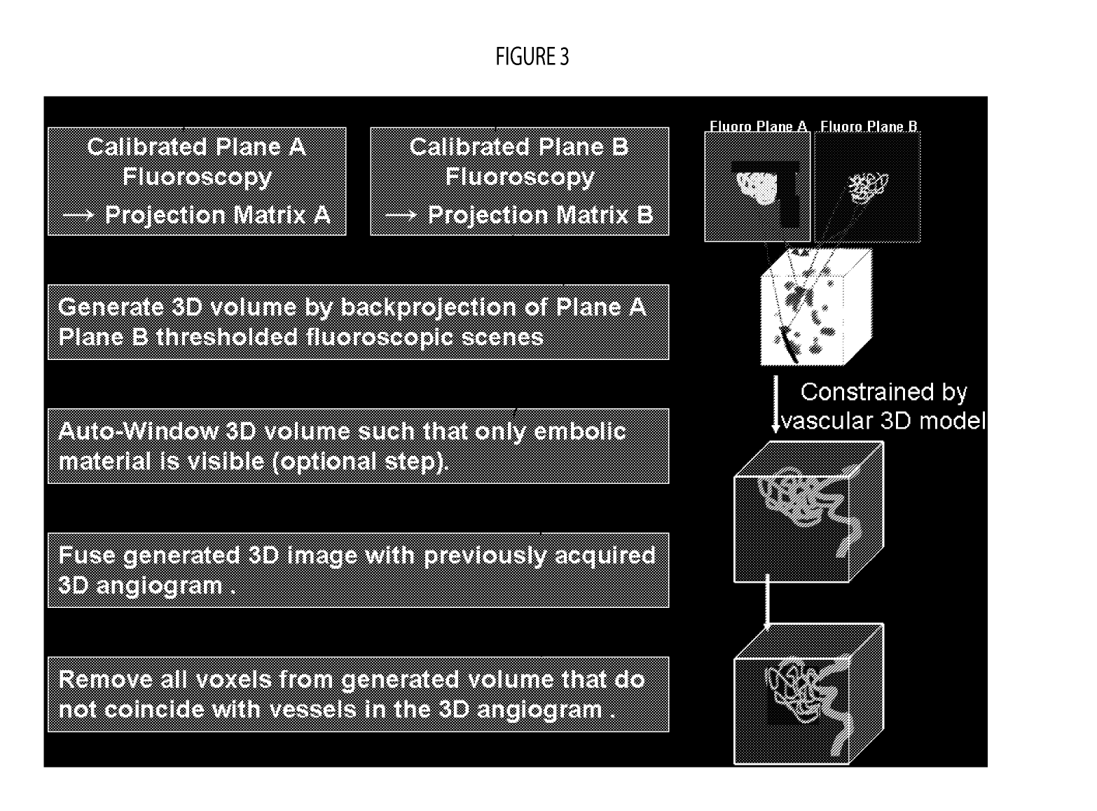

FIG. 3 shows a flowchart of a process for three dimensional (3D) visualization of embolic material using X-ray radiation imaging. As the radio-opaque embolic material is delivered, bi-plane fluoroscopic X-ray images 303 and 305 are acquired by system 10 from imaging device 25 by the two planes A and B and incorporated in projection matrices A and B respectively. Image data 25 in step 308 generates a 3D image dataset using X-ray images 303 and 305. In step 311, processor 15 advantageously (optionally) uses window leveling to provide a real-time 3D visualization of embolic material excluding other material. In step 314, processor 15 combines the generated 3D image dataset with a previously acquired 3D angiogram. Processor 15 in step 317 excludes voxels from the generated 3D image dataset that do not coincide with the 3D angiogram and constrains the dataset using the vascular angiogram model.

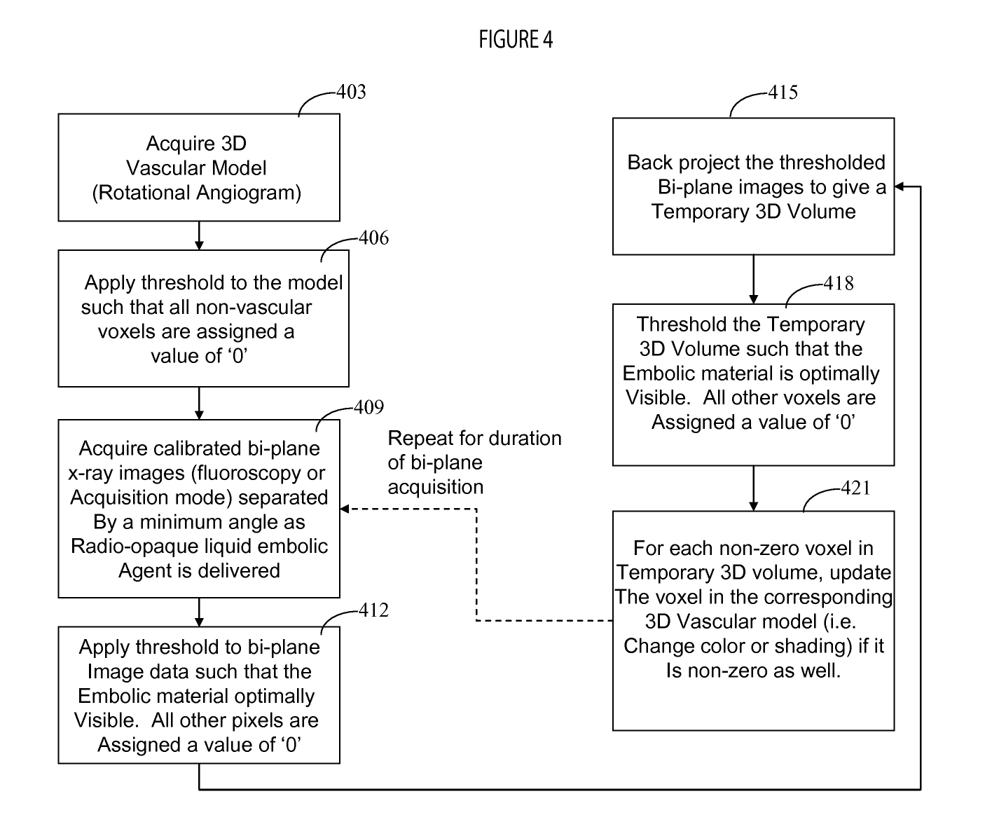

FIG. 4 shows a flowchart of a workflow process for three dimensional (3D) visualization of embolic material using X-ray radiation imaging. In step 403, Image data processor 15 acquires a 3D vascular model comprising a rotational angiogram. Processor 15 in step 406 applies a threshold to the model so that non-vascular voxels are assigned a value of ‘0’. In step 409 imaging system 25 acquires a calibrated bi-plane X-ray images (in fluoroscopy or Acquisition mode) separated by a minimum angle as radio-opaque liquid embolic agent is delivered. Processor 15 in step 412 applies a threshold to the acquired bi-plane image data such that the embolic material pixels are optimally visible and other pixels are assigned a value of ‘0’. In step 415 processor 15 back-projects the threshold bi-plane image data to provide a temporary 3D Volume. Processor 15 in step 418 applies a threshold to the temporary 3D Volume image data such that the embolic material is optimally visible and other voxels are assigned a value of ‘0’. In step 421, for each non-zero voxel in the temporary 3D volume image data, processor 15 updates the voxel in the corresponding 3D Vascular model (e.g., by changing color or shading) if it is non-zero as well.

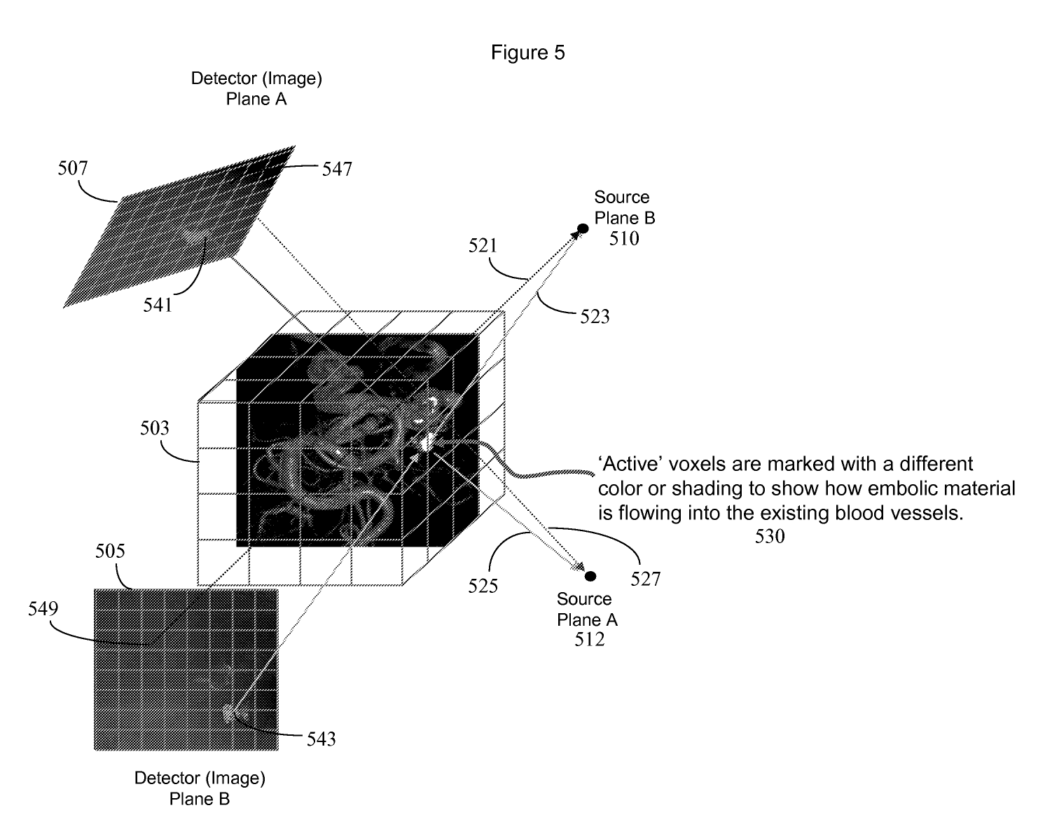

FIG. 5 illustrates identifying individual volume elements (e.g. element 530) in a 3D image dataset 503 corresponding to identified individual picture elements in 2D images (e.g. element 541 in image of plane 507 and element 543 in image of plane 505) representing embolic material. System 10 (FIG. 2) acquires the 3D image dataset 503 comprising a vascular model using rotational 3D angiography. The vascular model is thresholded (or segmented) such that any non-vascular voxels are given a value of ‘0’. The vascular model defines the possible space the embolic material can occupy (i.e., constrains the embolic material space). Image data processor 15 identifies an individual picture element (e.g. picture element 541) by detecting intersection of projected line 525 with one or more volume elements (e.g. voxel element 530) in the 3D image dataset or identifies an individual picture element (e.g. picture element 543) by detecting intersection of projected line 523 with one or more volume elements (e.g. voxel element 530) in the 3D image dataset.

Projected lines 521, 523, 525 and 527 represent backprojected rays from calibrated detector planes 505, 507 to corresponding X-ray sources 510 and 512. Projected lines such as lines 521 and 523 travel through 3D image dataset 503 from radiation source B (510) to corresponding points 549 and 543 of the image on radiation detector plane 505. Projected lines such as lines 525 and 527 travel through 3D image dataset 503 from radiation source A (512) to corresponding points 541 and 547 of the image on radiation detector plane 507. The bi-plane X-ray images of planes 505, 507 are acquired as the radio-opaque embolic material is delivered. Since the radio-opaque material has significantly higher X-ray attenuation properties than a skull and brain tissue, a thresholding function may be used to segment the embolic material in each of the X-ray images Non-embolic material pixels are assigned a value of ‘0’.

A non-filtered back projection of the two X-ray images is advantageously performed. Voxels in dataset 503 that have non-zero contributions from both planes are marked as active voxels. A logical ‘and’ operation is advantageously used to determine if the voxel in the original 3D vascular model contains the embolic material. If it does, the voxel is defined as an active voxel in a temporary volume. In this case, if a voxel is defined as non-zero in both the original 3D volume and the temporary 3D volume, the voxel is annotated (using color, shading or other visual attribute, for example) to show that it contains embolic material for that point in time. The result of this operation is a 3D vascular volume that is updated in real-time to show the location of the embolic material contained with the blood vessels. This obviates the need for the physician to switch his eyes between X-ray planes and removes the ambiguity of understanding where in 3D space the embolic material is going. Image data processor 15 identifies an active volume element (e.g. voxel element 530) in 3D image dataset 503 i.e., Active_Voxel=TRUE if an embolic material pixel(s) (e.g. pixel 541) from plane A 507 projects through a voxel (voxel 530) AND an embolic material pixel(s) (e.g. pixel 543) from plane 505 project through the voxel (voxel 530) AND the Voxel is included as a blood vessel in previously acquired 3D image dataset 503. Processor 15 marks an ‘Active’ voxel with a visual attribute (different color, shading, highlighting or other visual indication) to show how embolic material is flowing into the blood vessels.

System 10 enables use of real-time 3D image data to evaluate extent of liquid embolic deployment. The system 3D image model allows adjustment of a projection to an optimal working angle, regardless of the actual geometry of the angiographic planes. Additionally, many embolizations are performed in stages. Working from a 3D model enables a physician to plan and treat disease in a volumetric fashion, rather than rely on estimation from the 2D image projections. The system may also be used in a non real-time fashion using a single plane angiography system. Instead of simultaneously acquiring data from two planes, data is sequentially acquired from two projections separated by a minimum angular range. These image planes are used to update a 3D model provided during a latest intervention.

System 10 provides real-time 3D visualization of radio-opaque embolic materials using bi-plane fluoroscopic images, for example. High-contrast radio-opaque embolic material is segmented in planar fluoroscopic/roadmap images (subtracted fluoroscopic images) by thresholding, and high contrast pixels are back-projected to a 3D coordinate space. Alternatively, two fluoroscopic projections are backprojected into 3D space, and thresholding applied. Once in the 3D coordinate space, a 3D DSA (Digital Subtraction Angiography) angiogram of the vessel anatomy is used to constrain the back projected pixels to a previously rendered vessel tree, and the voxels that have an intersection from both planes are colored to show where the embolic material is present. This allows for fast, real-time 3D visualization of the deployment of radio-opaque embolic material using a previously acquired 3D DSA as the basis for rendering constraints.

The system 3D visualization advantageously enables a physician to use a working projection he needs to optimally view embolic treatment delivery. The working projection can be changed without moving a C-arm gantry. The system also renders multiple simultaneous 3D views at different working angles than can be achieved by C-arm X-ray planes. This enables greater viewing flexibility to a physician since he can also use 2D fluoroscopic images in addition to 3D visualization. The system constrains a back projection to a previously acquired 3D DSA of a target region. A physician views the exact progress of the current treatment without interference from a radio-opaque liquid embolic cast that exists from a previous treatment. This 3D visualization gives the physician a quick guide to the volume of the target that he has currently treated, which is difficult to assess using only 2D fluoroscopic images. The system reduces X-ray dosage given to a patient and operator, and shortens total procedure time.

System 10 (FIG. 2) in one embodiment generates a real-time rendering of radio-opaque embolic material in a 3D volume. A 3D Angiogram of the anatomy is acquired and registered with the current flow images. This registration is implicit if there is no additional patient movement from the time of the acquisition. Imaging system 25 acquires a biplane fluoroscopic image sequence while injecting the embolic material into the vessel. A threshold is applied to pixel luminance data of each fluoroscopic image frame so that radio-opaque material is primarily visible. A 3D volume is generated by backprojecting the thresholded and calibrated fluoroscopic image sequence into a new 3D volume. In a further embodiment, a window is applied to the new 3D volume and a threshold is applied to voxel luminance data in the windowed section (and the section is segmented) to ensure radio-opaque embolic material is visible in the image and other material is excluded and invisible. System 10 merges the new volume with the 3D angiogram so that only voxels that contain both vessels and contrast agent are visible. Other voxels are removed from the 3D dataset that includes backprojected bi-plane fluoroscopic data to provide a 3D vascular model. This vascular model is used to constrain reconstruction to voxels comprising vessels in which embolic material flows. The system advantageously enables fast, real-time 3D visualization of deployment of radio-opaque embolic material using a previously acquired 3D DSA as the basis for rendering constraints. The system improves understanding of the distribution of embolic material and the volumetric extent of the embolization in real-time.

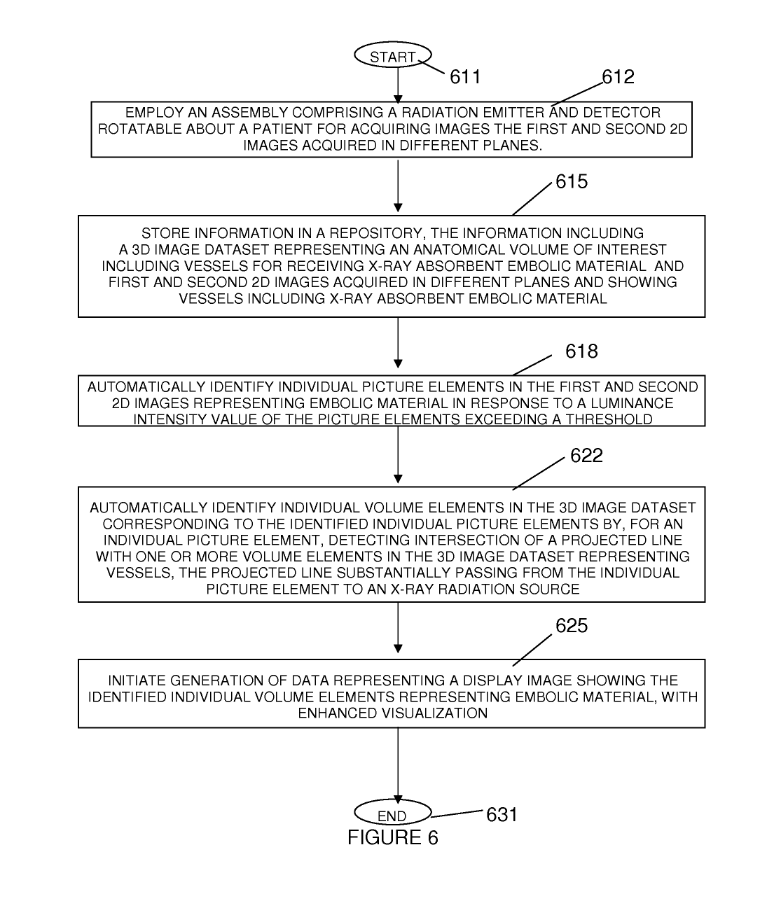

FIG. 6 shows a flowchart of a process employed by system 10 (FIG. 2) for three dimensional (3D) visualization of embolic material using X-ray radiation imaging. In step 612 following the start at step 611, image acquisition device 25 employs an assembly (e.g. a C-arm) comprising a radiation emitter and detector rotatable about a patient for acquiring first and second 2D images in different planes. In step 615, image data processor 15 stores information in repository 17. The information includes a 3D image dataset representing an anatomical volume of interest including vessels for receiving substantially radio-opaque X-ray absorbent embolic material and first and second 2D images acquired in different planes and showing vessels including X-ray absorbent embolic material

In step 618, image data processor 15 automatically identifies individual picture elements in the first and second 2D images representing embolic material in response to a luminance intensity value of the picture elements exceeding a threshold. In step 622, image data processor 15 automatically identifies individual volume elements in the 3D image dataset corresponding to the identified individual picture elements by, for an individual picture element, detecting intersection of a projected line with one or more volume elements in the 3D image dataset representing vessels, the projected line substantially passing from the individual picture element to an X-ray radiation source. Image data processor 15 automatically identifies the individual picture elements in response to a luminance value of the picture elements lying within a predetermined value range. Specifically, in one embodiment processor 15 automatically identifies the individual picture elements in response to a histogram associating the number of pixels in an image having a specific luminance intensity value with a range of available intensity values. In one embodiment the picture elements are pixels and the volume elements are voxels. Image data processor 15 in one embodiment applies a window to the 3D image dataset and applies a threshold to volume elements to ensure embolic material is visible in the display image and other material is excluded and invisible. Display processor 27 in step 625 initiates generation of data representing a display image showing the identified individual volume elements representing embolic material, with enhanced visualization. The process of FIG. 6 terminates at step 631.

A processor as used herein is a device for executing machine-readable instructions stored on a computer readable medium, for performing tasks and may comprise any one or combination of, hardware and firmware. A processor may also comprise memory storing machine-readable instructions executable for performing tasks. A processor acts upon information by manipulating, analyzing, modifying, converting or transmitting information for use by an executable procedure or an information device, and/or by routing the information to an output device. A processor may use or comprise the capabilities of a computer, controller or microprocessor, for example, and is conditioned using executable instructions to perform special purpose functions not performed by a general purpose computer. A processor may be coupled (electrically and/or as comprising executable components) with any other processor enabling interaction and/or communication there-between. Computer program instructions may be loaded onto a computer, including without limitation a general purpose computer or special purpose computer, or other programmable processing apparatus to produce a machine, such that the computer program instructions which execute on the computer or other programmable processing apparatus create means for implementing the functions specified in the block(s) of the flowchart(s). A user interface processor or generator is a known element comprising electronic circuitry or software or a combination of both for generating display elements or portions thereof. A user interface comprises one or more display elements enabling user interaction with a processor or other device.

An executable application, as used herein, comprises code or machine readable instructions for conditioning the processor to implement predetermined functions, such as those of an operating system, a context data acquisition system or other information processing system, for example, in response to user command or input. An executable procedure is a segment of code or machine readable instruction, sub-routine, or other distinct section of code or portion of an executable application for performing one or more particular processes. These processes may include receiving input data and/or parameters, performing operations on received input data and/or performing functions in response to received input parameters, and providing resulting output data and/or parameters. A graphical user interface (GUI), as used herein, comprises one or more display elements, generated by a display processor and enabling user interaction with a processor or other device and associated data acquisition and processing functions.

The UI also includes an executable procedure or executable application. The executable procedure or executable application conditions the display processor to generate signals representing the UI display images. These signals are supplied to a display device which displays the elements for viewing by the user. The executable procedure or executable application further receives signals from user input devices, such as a keyboard, mouse, light pen, touch screen or any other means allowing a user to provide data to a processor. The processor, under control of an executable procedure or executable application, manipulates the UI display elements in response to signals received from the input devices. In this way, the user interacts with the display elements using the input devices, enabling user interaction with the processor or other device. The functions and process steps herein may be performed automatically or wholly or partially in response to user command An activity (including a step) performed automatically is performed in response to executable instruction or device operation without user direct initiation of the activity. A histogram of an image is a graph that plots the number of pixels (on the y-axis herein) in the image having a specific intensity value (on the x-axis herein) against the range of available intensity values. The resultant curve is useful in evaluating image content and can be used to process the image for improved display (e.g. enhancing contrast).

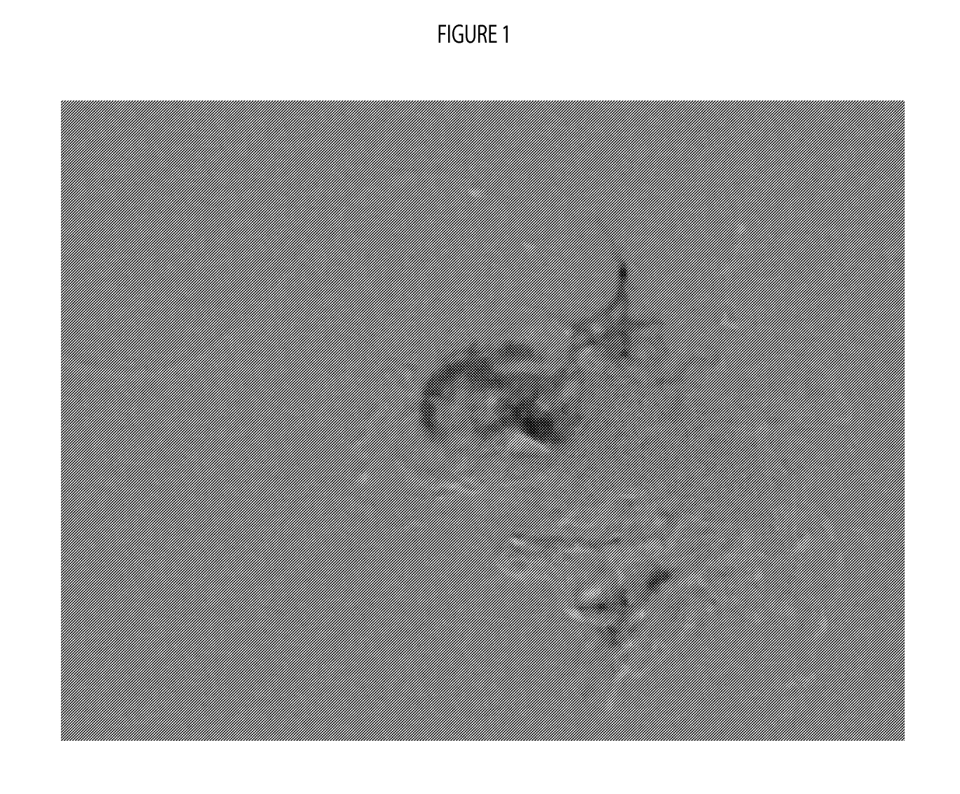

The system and processes of FIGS. 1-6 are not exclusive. Other systems, processes and menus may be derived in accordance with the principles of the invention to accomplish the same objectives. Although this invention has been described with reference to particular embodiments, it is to be understood that the embodiments and variations shown and described herein are for illustration purposes only. Modifications to the current design may be implemented by those skilled in the art, without departing from the scope of the invention. The system advantageously uses window leveling to back-project two fluoroscopic views of high-contrast material into a 3D volume to provide a real-time 3D visualization of embolic material comprising a constrained reconstruction of a windowed 3D volume as a 3D Angiogram. Further, the processes and applications may, in alternative embodiments, be located on one or more (e.g., distributed) processing devices on a network linking the units FIG. 1. Any of the functions and steps provided in FIGS. 1-6 may be implemented in hardware, software or a combination of both. No claim element herein is to be construed under the provisions of 35 U.S.C. 112, sixth paragraph, unless the element is expressly recited using the phrase “means for.”

What is claimed is:

1. A system for three dimensional (3D) visualization of embolic material using X-ray radiation imaging, comprising:
a repository of information including
a 3D image dataset representing an anatomical volume of interest including vessels for receiving X-ray absorbent embolic material and
first and second 2D images acquired in different planes and showing vessels including X-ray absorbent embolic material;
an image data processor for automatically,
identifying individual picture elements in said first and second 2D images representing embolic material in response to a luminance intensity value of the picture elements exceeding a threshold and
identifying individual volume elements in said 3D image dataset corresponding to the identified individual picture elements; and
a display processor for initiating generation of data representing a display image showing the identified individual volume elements representing embolic material, with enhanced visualization.

2. A system according to claim 1, wherein
said image data processor automatically identifies individual volume elements in said 3D image dataset corresponding to the identified individual picture elements by, for an individual picture element, detecting intersection of a projected line with one or more volume elements in said 3D image dataset representing vessels, said projected line substantially passing from said individual picture element to an X-ray radiation source.

3. A system according to claim 1, wherein
said picture elements are pixels and
said volume elements are voxels.

4. A system according to claim 1, wherein
said X-ray absorbent embolic material is substantially radio-opaque.

5. A system according to claim 1, wherein
said image data processor automatically identifies said individual picture elements in response to a histogram associating the number of pixels in an image having a specific luminance intensity value with a range of available intensity values.

6. A system according to claim 1, wherein
said image data processor automatically identifies said individual picture elements in response to a luminance value of the picture elements lying within a predetermined value range

7. A system according to claim 1, including
an image acquisition device including an assembly comprising a radiation emitter and detector rotatable about a patient for acquiring said first and second 2D images in different planes.

8. A system according to claim 1, wherein
said image data processor applies a window to said 3D image dataset and applies a threshold to volume elements to ensure embolic material is visible in said display image and other material is excluded and invisible.

9. A method for three dimensional (3D) visualization of embolic material using X-ray radiation imaging, comprising the activities of:
storing information in a repository of information, said information including
a 3D image dataset representing an anatomical volume of interest including vessels for receiving X-ray absorbent embolic material and
first and second 2D images acquired in different planes and showing vessels including X-ray absorbent embolic material;
automatically identifying individual picture elements in said first and second 2D images representing embolic material in response to a luminance intensity value of the picture elements exceeding a threshold;
identifying individual volume elements in said 3D image dataset corresponding to the identified individual picture elements; and
initiating generation of data representing a display image showing the identified individual volume elements representing embolic material, with enhanced visualization.

10. A method according to claim 9, including the activity of
automatically identifying individual volume elements in said 3D image dataset corresponding to the identified individual picture elements by, for an individual picture element, detecting intersection of a projected line with one or more volume elements in said 3D image dataset representing vessels, said projected line substantially passing from said individual picture element to an X-ray radiation source.

11. A method according to claim 9, wherein
said picture elements are pixels and
said volume elements are voxels.

12. A method according to claim 9, wherein
said X-ray absorbent embolic material is substantially radio-opaque.

13. A method according to claim 9, including the activity of
automatically identifying said individual picture elements in response to a histogram associating the number of pixels in an image having a specific luminance intensity value with a range of available intensity values.

14. A method according to claim 9, including the activity of
automatically identifying said individual picture elements in response to a luminance value of the picture elements lying within a predetermined value range

15. A method according to claim 9, including the activity of
employing an assembly comprising a radiation emitter and detector rotatable about a patient for acquiring images said first and second 2D images acquired in different planes.

16. A method according to claim 9, including the activity of
applying a window to said 3D image dataset and applies a threshold to volume elements to ensure embolic material is visible in said display image and other material is excluded and invisible.

17. A system for three dimensional (3D) visualization of embolic material using X-ray radiation imaging, comprising:
a repository of information including
a 3D image dataset representing an anatomical volume of interest including vessels for receiving X-ray absorbent embolic material and
first and second 2D images acquired in different planes and showing vessels including X-ray absorbent embolic material;
an image data processor for automatically,
identifying individual picture elements in said first and second 2D images representing embolic material in response to a luminance intensity value of the picture elements exceeding a threshold and
identifying individual volume elements in said 3D image dataset corresponding to the identified individual picture elements by, for an individual picture element, detecting intersection of a projected line with one or more volume elements in said 3D image dataset representing vessels, said projected line substantially passing from said individual picture element to an X-ray radiation source; and
a display processor for initiating generation of data representing a display image showing the identified individual volume elements representing embolic material, with enhanced visualization.
